(19) United States
(12) Patent Application Publication
(10) Pub. No.: US 2025/0302373 A1
(43) Pub. Date: Oct. 2, 2025

(54) ARTIFICIAL INTELLIGENCE-BASED JOINT FUNCTION ANALYSIS AND MONITORING DEVICE AND CONTROL METHOD THEREFOR

(71) Applicant: IT-PHY INC., Seoul (KR)

(72) Inventors: Sung Min RHEE, Seoul (KR); Jae Jun LEE, Seongnam-si (KR)

(73) Assignee: IT-PHY INC., Seoul (KR)

(21) Appl. No.: 19/239,022

(22) Filed: Jun. 16, 2025

Related U.S. Application Data
(63) Continuation of application No. PCT/KR2023/095117, filed on Dec. 15, 2023.

(30) Foreign Application Priority Data
Dec. 15, 2022 (KR) 10-2022-0176088
Dec. 14, 2023 (KR) 10-2023-0181712

(51) Int. Cl. A61B 5/00; A61B 6/46; G06T 7/70; G16H 10/20; G16H 20/30

(52) U.S. Cl. CPC A61B 5/4528; A61B 6/465; G06T 7/70; G16H 10/20; G16H 20/30

(57) ABSTRACT

The present disclosure relates to an AI-based joint function analyzing and monitoring device that provides a life-cycle health management service for a user by converging IT technologies for the prevention and management of musculoskeletal diseases, provides a comparison analysis of the user's current joint status and symptom similarity with those with diseases by using only data of patients with diseases diagnosed by professors at tertiary university hospitals without image analysis results such as X-rays and MRIs, and operates as an auxiliary tool for doctors to diagnose diseases.

### CROSS-REFERENCE TO RELATED APPLICATIONS

The present application is a continuation of International Patent Application No. PCT/KR2023/095117, filed on Dec. 15, 2023, which is based upon and claims the benefit of priority to Korean Patent Application Nos. 10-2022-0176088 filed on Dec. 15, 2022 and 10-2023-0181712 filed on Dec. 14, 2023. The disclosures of the above-listed applications are hereby incorporated by reference herein in their entirety.

### BACKGROUND

Embodiments of the present disclosure described herein relate to a joint function analyzing and monitoring device, and more particularly, relate to an artificial intelligence (AI)-based joint function analyzing and monitoring device and a control method therefor.

Nowadays, in addition to the International Classification of Diseases, a classification system for orthopedics is organized based on musculoskeletal diagnosis and statistics. Diagnosis according to the classification system is based on a combination of records and tests, including an interview with an orthopedic surgeon, reading of photographs, and surveys.

In the prior art, to measure a patient's joint range of motion angles, a primitive protractor is used or a visual estimate is made. Compared to the prior art, a more advanced technology refers to a joint angle measurement system based on sensors, and measures angles by attaching sensors to upper and lower arms and chest. However, the technology is based on Bluetooth. Accordingly, there is a possibility of harm from electromagnetic waves, and wearing the sensor is also very cumbersome.

In addition, in the conventional technology, even when a functional evaluation of joint status is conducted, each patient checks his/her own condition on A4 paper, and then additional staff transfer the checked data back to an EMR computer system, which is very inefficient in terms of manpower utilization.

Besides, although personal health data on patients is collected at each hospital, data collection is inconsistent, and thus the data is scattered. Accordingly, because big data collection is impossible, patients go through unnecessary procedures of receiving the same re-examination when visiting different hospitals, and patients may only connect with medical staff when they receive treatment in person at the hospital, and users feel inconvenienced because prevention before treatment or monitoring between treatments is impossible.

### SUMMARY

Embodiments of the present disclosure provide an AI-based joint function analyzing and monitoring device that provides a life-cycle health management service for a user by converging IT technologies for the prevention and management of musculoskeletal diseases, provides a comparison analysis of the user's current joint status and symptom similarity with those with diseases by using only data of patients with diseases diagnosed by professors at tertiary university hospitals without image analysis results such as X-rays and MRIs, and operates as an auxiliary tool for doctors to diagnose diseases.

Embodiments of the present disclosure provide an AI-based joint function analyzing and monitoring device that compares the current symptoms of users with those of existing patients with diseases, delivers the similarity, lists possible diseases, and recommends that the users go to a nearby hospital for diagnosis.

Embodiments of the present disclosure provide an AI-based joint function analyzing and monitoring device that may complement existing inaccurate joint range measurement technology to provide AI-based accurate joint range measurement.

Embodiments of the present disclosure provide an AI-based joint function analyzing and monitoring device that automatically transmits the evaluated results to an EMR computer system when a user self-evaluates his or her joint status.

Embodiments of the present disclosure provide an AI-based joint function analyzing and monitoring device that may suggest personalized rehabilitation exercises of a healthcare provider based on the transmitted data.

Embodiments of the present disclosure provide an AI-based joint function analyzing and monitoring device that may provide a common form of a joint evaluation tool to facilitate data sharing between hospitals, and may collect and manage data on a single server.

Embodiments of the present disclosure provide an AI-based joint function analyzing and monitoring device that may provide a tool of monitoring recovery progress before and during the examination in a hospital.

Problems to be solved by the present disclosure are not limited to the problems mentioned above, and other problems not mentioned will be clearly understood by those skilled in the art from the following description.

According to an embodiment, an AI-based joint function analyzing and monitoring device includes an input module that receives a user input, a display module that displays a graphic image, a memory that stores at least one process for performing an operation and stores a user input and data, a camera module that captures an image in front, and a processor that performs the AI-based joint function analysis operation according to the process. The processor allows the display module to display a start screen for receiving user login from a user, displays a menu screen for receiving an entire service including joint measurement, a mood state check, and AI-recommended exercise of the user, displays a joint measurement screen when receiving a joint measurement input from a user, captures a joint image of the user taking a plurality of poses on the joint measurement screen by using the camera module, displays a survey screen for a joint state of the user, and receive an input for a survey from a user, displays an image for displaying a pain area of the user corresponding to an answer to the survey, and receives an input for the pain area from a user, infers a state of the user based on the joint image, a result of the survey, and a result of the pain area, and displays the state of the user.

In the AI-based joint function analyzing and monitoring device according to an embodiment of the present disclosure, when the joint image is a two-dimensional image, the processor extracts a pose of a three-dimensional image based on the joint image and analyzes a joint angle of the three-dimensional image.

In the AI-based joint function analyzing and monitoring device according to an embodiment of the present disclosure, the processor identifies a degree of pain through the survey, and identifies a pose in which pain occurs, by asking a question about an action matching a direction in which the joint corresponding to the joint image moves.

In the AI-based joint function analyzing and monitoring device according to an embodiment of the present disclosure, the processor displays a screen for searching for at least one of a doctor and a hospital corresponding to the pain area, and performs a search when receiving an input of a user.

In the AI-based joint function analyzing and monitoring device according to an embodiment of the present disclosure, the processor displays a screen for suggesting a customized exercise suggested by a doctor corresponding to the state of the user.

In the AI-based joint function analyzing and monitoring device according to an embodiment of the present disclosure, when receiving a user selection input, the processor displays a customized exercise image suggested by the doctor.

In the AI-based joint function analyzing and monitoring device according to an embodiment of the present disclosure, the processor displays doctor information corresponding to the state of the user, displays a joint range corresponding to the joint image, and displays a record screen for identifying feedback from a doctor corresponding to the doctor information.

In the AI-based joint function analyzing and monitoring device according to an embodiment of the present disclosure, the processor displays a screen for showing a treatment and an appointment schedule of the user.

In the AI-based joint function analyzing and monitoring device according to an embodiment of the present disclosure, the processor displays a chat execution screen for chatting with the user.

According to an embodiment, an AI-based joint function analyzing and monitoring control method includes displaying a start screen for receiving user login from a user, displaying a menu screen for receiving an entire service including joint measurement, a mood state check, and AI-recommended exercise of the user, displaying a joint measurement screen when receiving a joint measurement input from a user, capturing a joint image of the user taking a plurality of poses on the joint measurement screen by using a camera, displaying a survey screen for a joint state of the user, and receiving an input for a survey from a user, displaying an image for displaying a pain area of the user corresponding to an answer to the survey, and receiving an input for the pain area from a user, inferring a state of the user based on the joint image, a result of the survey, and a result of the pain area, and displaying the state of the user.

Besides, a computer program stored in a computer-readable recording medium for executing a method to implement the present disclosure may be further provided.

In addition, a computer-readable recording medium for recording a computer program for performing the method for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a configuration diagram of an AI-based joint function analyzing and monitoring device, according to an embodiment of the present disclosure;

FIG. 2 is a diagram illustrating a flowchart of an AI-based joint function analyzing and monitoring method, according to an embodiment of the present disclosure;

FIG. 3 is a diagram illustrating a start screen of an AI-based joint function analyzing and monitoring device, according to an embodiment of the present disclosure;

FIG. 4 is a drawing for describing a function of a start screen icon, according to an embodiment of the present disclosure;

FIG. 5 is a drawing illustrating a menu screen, according to an embodiment of the present disclosure;

FIG. 6 is a diagram illustrating a joint-state evaluating screen, according to an embodiment of the present disclosure;

FIG. 7 is a diagram illustrating an example of a joint measuring screen (camera setting), according to an embodiment of the present disclosure;

FIG. 8 is a drawing illustrating an embodiment of a joint measuring screen, according to an embodiment of the present disclosure;

FIG. 9 is a drawing illustrating an embodiment of a joint measuring screen, according to an embodiment of the present disclosure;

FIG. 10 is a drawing illustrating an example of a survey screen, according to an embodiment of the present disclosure;

FIG. 11 is a drawing illustrating an embodiment of a pain area screen, according to an embodiment of the present disclosure;

FIG. 12 is a drawing illustrating an example of a survey screen, according to an embodiment of the present disclosure;

FIG. 13 is a diagram illustrating an embodiment of a state screen of a user, according to an embodiment of the present disclosure;

FIG. 14 is a diagram illustrating an example of a search screen, according to an embodiment of the present disclosure;

FIG. 15 is a diagram illustrating an embodiment of a doctor information screen, according to an embodiment of the present disclosure;

FIG. 16 is a diagram illustrating an embodiment of a doctor treatment and customized exercise suggestion screen, according to an embodiment of the present disclosure;

FIG. 17 is a diagram illustrating an example of a doctor suggestion motion screen, according to an embodiment of the present disclosure;

FIG. 18 is a diagram illustrating an embodiment of a doctor feedback record screen, according to an embodiment of the present disclosure;

FIG. 19 is a diagram illustrating an embodiment of My-Page, according to an embodiment of the present disclosure;

FIG. 20 is a drawing illustrating an example of a chat execution screen, according to an embodiment of the present disclosure;

FIG. 21 is a drawing illustrating an embodiment of an operating system, according to an embodiment of the present disclosure;

FIG. 22 is a drawing illustrating an example of a main description of an operating system, according to an embodiment of the present disclosure;

FIG. 23 is a diagram illustrating a software structure, according to an embodiment of the present disclosure;

FIG. 24 is a diagram illustrating a main function of a software structure, according to an embodiment of the present disclosure;

FIG. 25 is a diagram illustrating a software algorithm, according to an embodiment of the present disclosure;

FIG. 26 is a diagram illustrating a comparison group and a functional recovery speed analysis, according to an embodiment of the present disclosure;

FIG. 27 is a diagram illustrating an example of predicting a disease through comparison with other patients, according to an embodiment of the present disclosure;

FIG. 28 is a diagram illustrating state analysis of a user, according to an embodiment of the present disclosure; and

FIG. 29 is a diagram illustrating a configuration of an AI-based joint function analyzing and monitoring device, according to an embodiment of the present disclosure.

### DETAILED DESCRIPTION

The same reference numerals denote the same elements throughout the present disclosure. The present disclosure does not describe all elements of embodiments. Well-known content in a technical field, to which the present disclosure belongs, or redundant content in which embodiments are the same as one another will be omitted. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

In this specification, the present disclosure may be implemented not only as a server system but also as various devices capable of performing computational processing and providing results to a user. For example, the present disclosure may include all of a computer, a server device, and a portable terminal, or may be in any one form.

Here, for example, the computer may include a notebook computer, a desktop computer, a laptop computer, a tablet PC, a slate PC, and the like, which are equipped with a web browser.

The server device may be a server that processes information by communicating with an external device and may include an application server, a computing server, a database server, a file server, a game server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet (WiBro) terminal, and a wearable device such as a timepiece, a ring, a bracelet, an anklet, a necklace, glasses, a contact lens, or a head-mounted device (HMD).

Functions related to artificial intelligence according to an embodiment of the present disclosure are operated through a processor and a memory. The processor may consist of one or more processors. In this case, the one or more processors may be a general-purpose processor (e.g., a CPU, an AP, or a digital signal processor (DSP)), a graphics-dedicated processor (e.g., a GPU or a vision processing unit (VPU)), or an artificial intelligence (AI)-dedicated processor (e.g., an NPU). Under control of the one or more processors, input data may be processed depending on an AI model, or a predefined operating rule stored in the memory. Alternatively, when the one or more processors are AI-dedicated processors, the AI-dedicated processor may be designed with a hardware structure specialized for processing a specific AI model.

The predefined operating rule or the artificial intelligence model is created through learning. Here, being created through learning means creating the predefined operating rule or the artificial intelligence model configured to perform desired features (or purposes) as a basic artificial intelligence model is learned by using pieces of learning data by a learning algorithm. This learning may be performed by a device itself, on which the artificial intelligence according to an embodiment of the present disclosure is performed, or may be performed through a separate server and/or system. For example, the learning algorithm may include supervised learning, unsupervised learning, semi-supervised learning, or reinforcement learning, but may not be limited to the above example.

An artificial intelligence model may be composed of a plurality of neural network layers. The plurality of neural network layers respectively have a plurality of weight values, and each of the plurality of neural network layers performs neural network calculation through calculations between the calculation result of the previous layer and the plurality of weight values. The plurality of weight values of the plurality of neural network layers may be optimized by the learning result of the artificial intelligence model. For example, during a learning process, the plurality of weight values may be updated such that a loss value or a cost value obtained from the artificial intelligence model is reduced or minimized. The artificial neural network may include a deep neural network (DNN). The artificial neural network may be, for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited to the above-described example.

The processor may create a neural network, may train or learn a neural network, or may perform operations based on received input data, and then may generate an information signal or may retrain the neural network based on the performed results.

It will be understood by those skilled in the art that a neural network may include any neural network, but is not limited to a convolutional neural network (CNN), a recurrent neural network (RNN), a perceptron, a multilayer perceptron, a feed forward (FF), a radial basis network (RBF), a deep feed forward (DFF), a long short term memory (LSTM), a gated recurrent unit (GRU), an auto encoder (AE), a variational auto encoder (VAE), a denoising auto encoder (DAE), a sparse auto encoder (SAE), a Markov chain (MC), a Hopfield network (HN), a Boltzmann machine (BM), a restricted Boltzmann machine (RBM), a deep belief network (DBN), a deep convolutional network (DCN), a deconvolutional network (DN), a deep convolutional inverse graphics network (DCIGN), a generative adversarial network (GAN), a liquid state machine (LSM), an extreme learning machine (ELM), an echo state network (ESN), a deep residual network (DRN), a differentiable neural computer (DNC), a neural turning machine (NTM), a capsule network (CN), a Kohonen network (KN), and an attention network (AN).

According to an embodiment of the present disclosure, the processor may use various artificial intelligence structures and algorithms such as a convolution neural network (CNN) (e.g., GoogleNet, AlexNet, or VGG Network), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) Network, a classification network, Generative Modeling, eXplainable AI, Continual AI, Representation Learning, AI for Material Design, algorithms for natural language processing (e.g., BERT, SP-BERT, MRC/QA, Text Analysis, Dialog System, GPT-3, and GPT-4), algorithms for vision processing (e.g., Visual Analytics, Visual Understanding, Video Synthesis, and ResNet), algorithms for data intelligence (e.g., Anomaly Detection, Prediction, Time-Series Forecasting, Optimization, Recommendation, and Data Creation), but is not limited thereto. Hereinafter, an embodiment of the present disclosure will be described in detail with reference to the accompanying drawings.

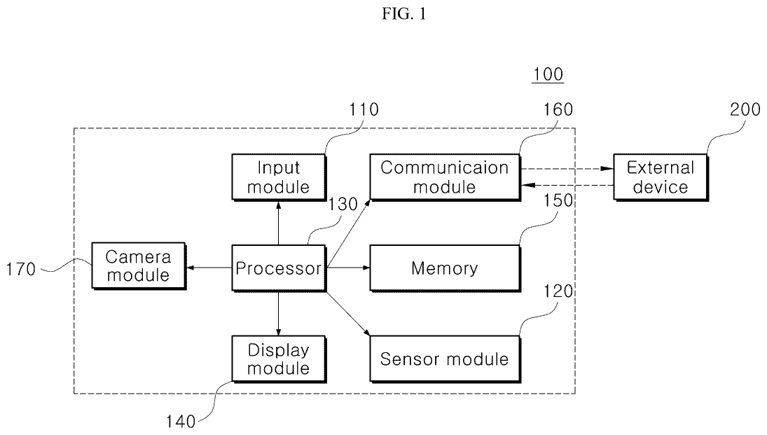

FIG. 1 is a configuration diagram of an AI-based joint function analyzing and monitoring device, according to an embodiment of the present disclosure.

Referring to FIG. 1, an AI-based joint function analyzing and monitoring device 100 includes an input module 110, a sensor module 120, a processor 130, a display module 140, a memory 150, a communication module 160, and a camera module 170.

The input module 110 receives a user input.

The sensor module 120 senses joint movement.

The processor 130 performs an AI-based joint function analysis operation according to the process.

The processor 130 allows the display module 140 to display a start screen for receiving user login from a user, displays a menu screen for receiving the entire service including joint measurement, a mood state check, and an AI-recommended exercise of the user, displays a joint measurement screen when the processor 130 receives a joint measurement input from the user, captures a joint image of the user taking a plurality of poses on the joint measurement screen by using the camera module 170, displays a survey screen for a joint state of the user, receives an input for a survey from the user, displays an image for displaying a pain area of the user corresponding to an answer to the survey, receives an input for the pain area from the user, infers a state of the user based on the joint image, a result of the survey, and a result of the pain area, and displays the state of the user.

The display module 140 displays a graphic image.

The memory 150 stores at least one process for performing operations and stores user inputs and data.

The communication module 160 exchanges data with an external device 200.

Here, the external device 200 includes external devices such as smartphones, PCs, laptops, tablet PCs, and the like.

The camera module 170 captures images positioned in front.

The camera module 170 photographs a subject positioned in front according to a control command from the processor 130.

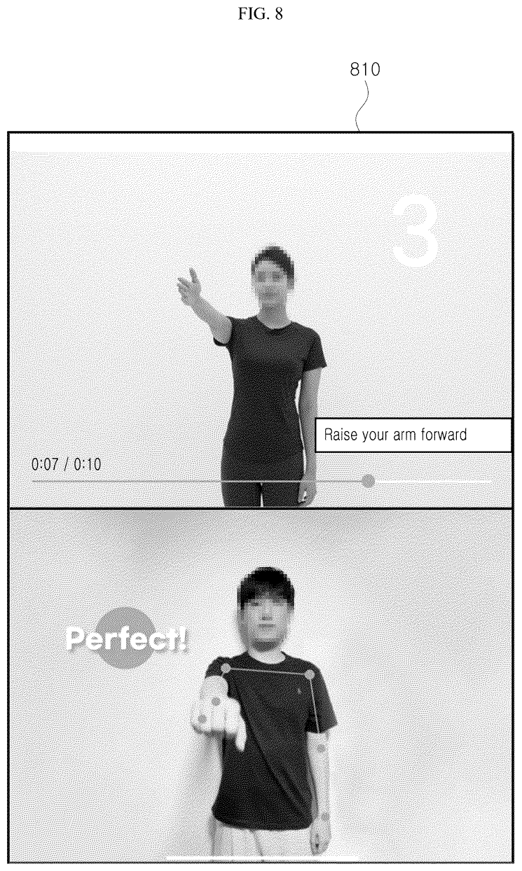

When the joint image is a two-dimensional image, the processor 130 extracts the pose of a three-dimensional image based on the joint image and analyzes the joint angle of the three-dimensional image. Detailed descriptions of this are given in FIG. 8.

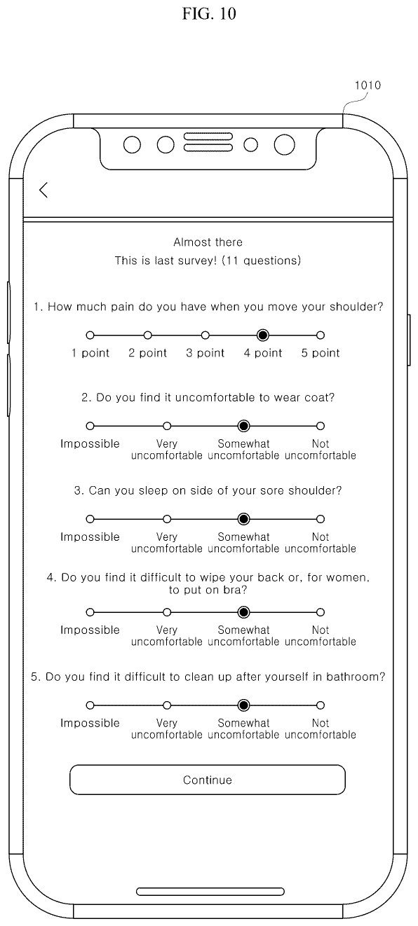

The processor 130 identifies the degree of pain through the survey, and determines a pose in which the pain occurs, by asking questions about actions matching a direction in which the joint corresponding to the joint image moves. Detailed descriptions of this are given in FIG. 10.

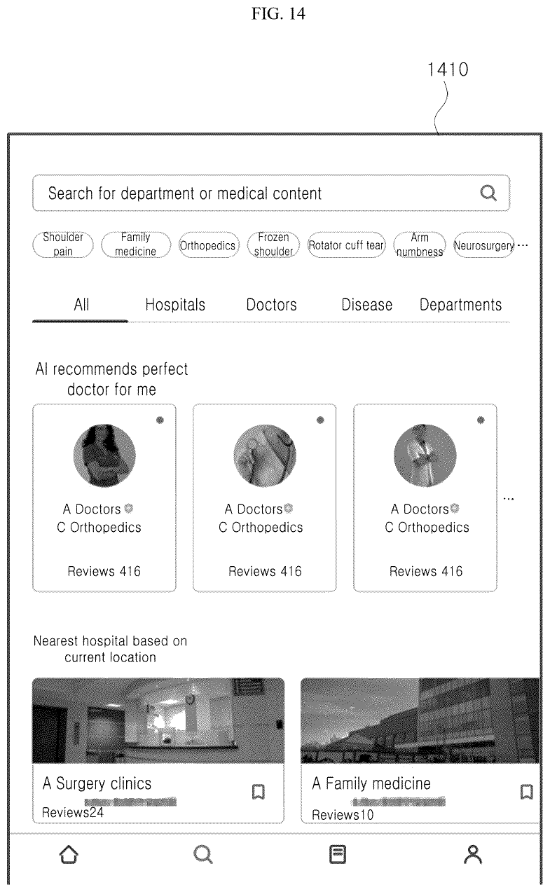

The processor 130 displays a screen for searching for at least one of a doctor or a hospital corresponding to the pain area, and performs a search when receiving an input of the user. Detailed descriptions of this are given in FIG. 14.

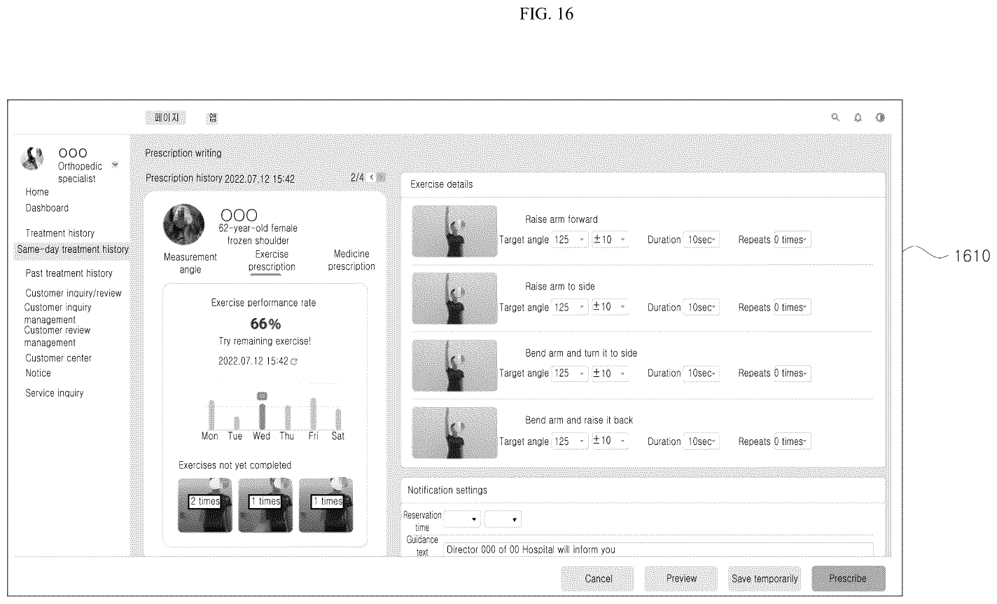

The processor 130 displays a screen for suggesting a customized exercise suggested by a doctor corresponding to the user's state. Detailed descriptions of this are given in FIG. 16.

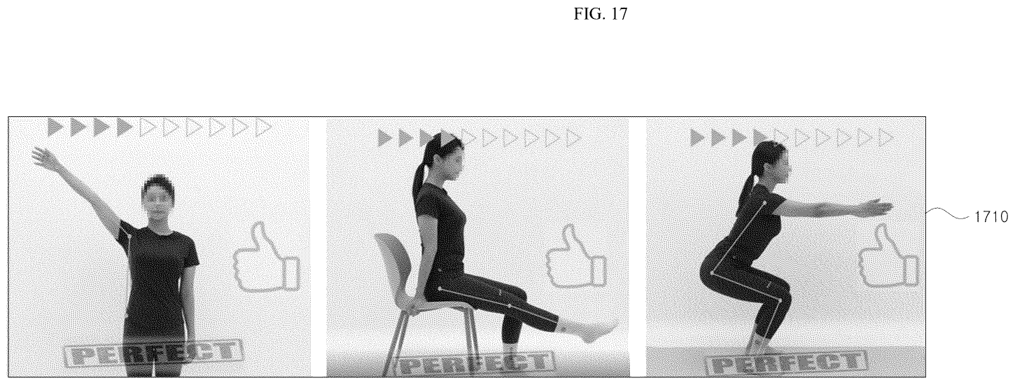

When receiving a user selection input, the processor 130 displays the customized exercise image suggested by the doctor. Detailed descriptions of this are given in FIG. 17.

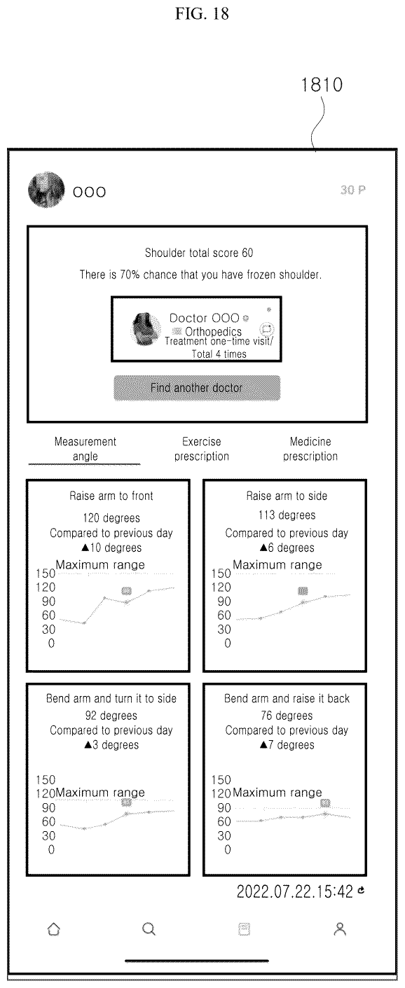

The processor 130 displays doctor information corresponding to the user's state, displays a joint range corresponding to the joint image, and displays a record screen for identifying feedback from the doctor corresponding to the doctor information. Detailed descriptions of this are given in FIG. 18.

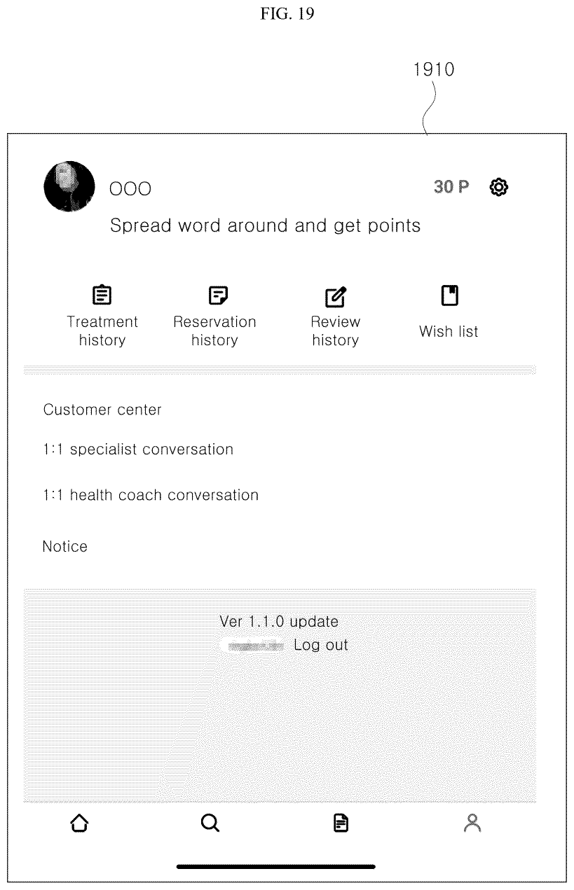

The processor 130 displays a screen for showing the user's treatment and appointment schedule. Detailed descriptions of this are given in FIG. 19.

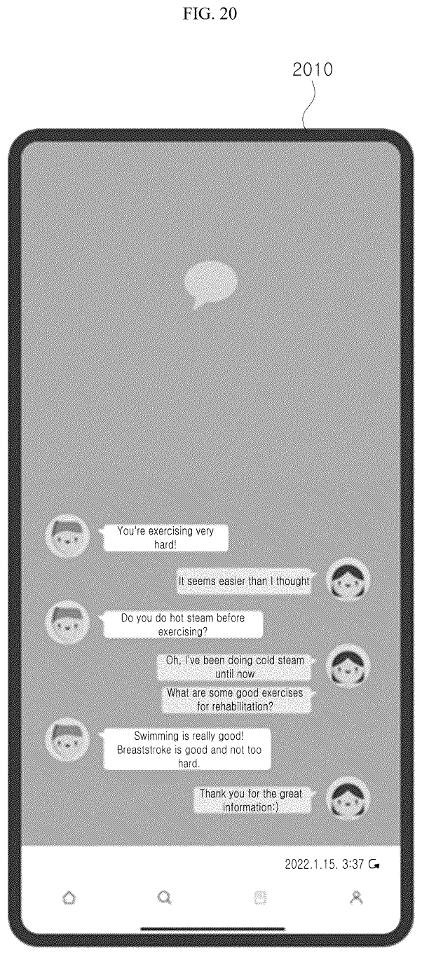

The processor 130 displays a chat execution screen for chatting with the user. Detailed descriptions of this are given in FIG. 20.

The processor 130 receives detailed information including the user's gender and age, calculates a comparison target based on the detailed information, and displays a functional recovery speed comparing and analyzing result of comparing the calculated comparison target with the functional recovery speed.

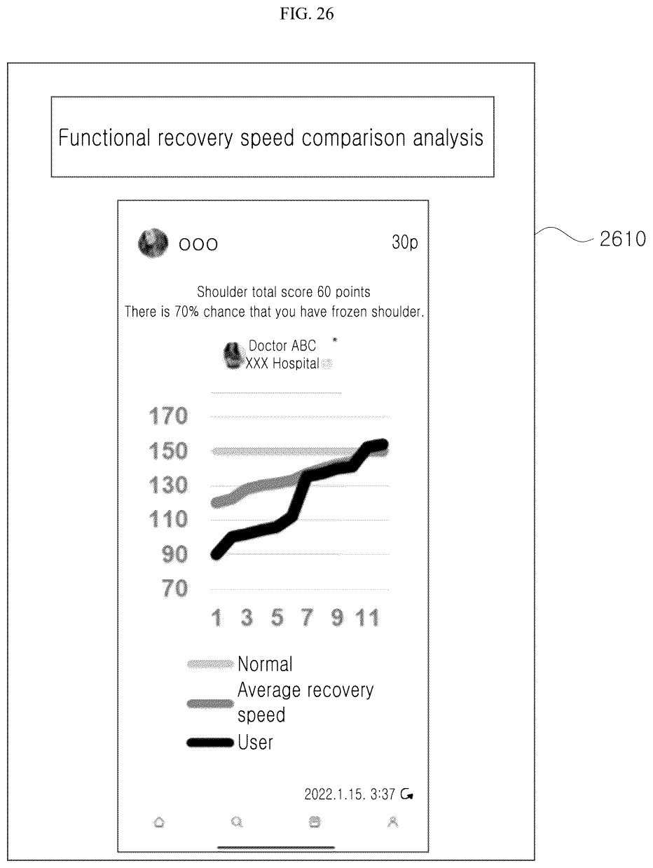

Detailed descriptions of this are given in FIG. 26.

Although the present disclosure is described with a focus on a shoulder, it may be applied to various joints such as knees or spinal joints in addition to the shoulder.

However, the components shown in FIG. 1 are not essential in implementing the present disclosure. The present disclosure described herein may have more or fewer components than those listed above.

The communication module 160 may include one or more components capable of communicating with an external device, and may include, for example, at least one of a broadcast reception module, a wired communication module, a wireless communication module, a short-range communication module, and a location information module.

The input module 110 may be used to enter image information (or signal), audio information (or signal), data, or information entered by a user. The input module 110 may include at least one of at least one camera, at least one microphone, and a user input unit. Speech data or image data collected by the input module 110 may be analyzed and processed as a control command of a user.

The display module 140 displays (outputs) information processed in the present disclosure. For example, the present disclosure may display execution screen information of a running application program (e.g., an application), or a user interface (UI) or graphical user interface (GUI) information according to such the execution screen information.

The memory 150 may store data for supporting various functions of the present disclosure, and a program for operations of a controller, may store pieces of input/output data (e.g., music files, still images, videos, and the like), and may store a plurality of application programs (or applications) running on the AI-based joint function analyzing and monitoring device 100, pieces of data for operations of the present disclosure, and instructions of the present disclosure. At least part of the application programs may be downloaded from an external server through wireless communication.

The memory 150 may include the type of a storage medium of at least one of a flash memory type, hard disk type, a solid state disk (SSD) type, a silicon disk drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc. Furthermore, the memory 150 may be separate from the present disclosure, but may be a database connected by wire or wirelessly, and may be implemented as a database system.

The processor 130 may include at least one core, and may be implemented with a memory that stores data regarding an algorithm for controlling operations of components, or a program for implementing the algorithm, and the at least one processor (not illustrated) that perform the above-described operation by using the data stored in the memory. At this time, the memory and the processor may be implemented as separate chips. Alternatively, the memory and the processor may be implemented as a single chip.

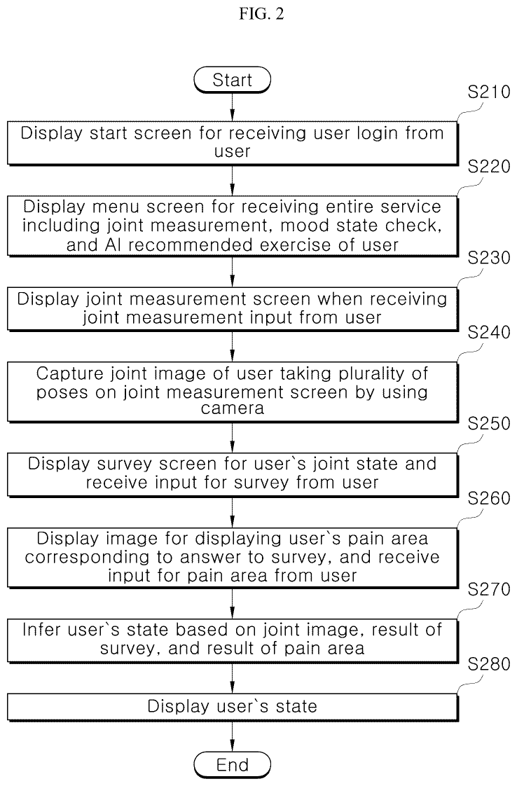

Furthermore, the processor 130 may control one of the components described above or the combination of the components to implement various embodiments of the present disclosure described below with reference to FIGS. 2 to 29 on the present disclosure.

At least one component may be added or deleted to correspond to the performance of the components illustrated in FIG. 1. Furthermore, it will be easily understood by those skilled in the art that mutual locations of the components may be changed to correspond to the performance or structure of the system.

In the meantime, each component shown in FIG. 1 refers to software components and/or hardware components such as field programmable gate array (FPGA) and application specific integrated circuit (ASIC).

FIG. 2 is a diagram illustrating a flowchart of an AI-based joint function analyzing and monitoring method, according to an embodiment of the present disclosure. The present disclosure is performed by the AI-based joint function analyzing and monitoring device 100 or the processor 130.

Referring to FIG. 2, it displays a start screen for receiving user login from a user (S210).

It displays a menu screen for receiving an entire service including joint measurement, a mood state check, and an AI recommended exercise of the user (S220).

When receiving a joint measurement input from the user, it displays a joint measurement screen (S230).

It captures a joint image of a user taking a plurality of poses on the joint measurement screen by using a camera (S240).

It displays a survey screen for the user's joint state and receives an input for the survey from the user (S250).

It displays an image for displaying the user's pain area corresponding to the answer to the survey, and receives an input for the pain area from the user (S260).

It infers the user's state based on the joint image, the result of the survey, and the result of the pain area (S270).

It displays the user's state (S280).

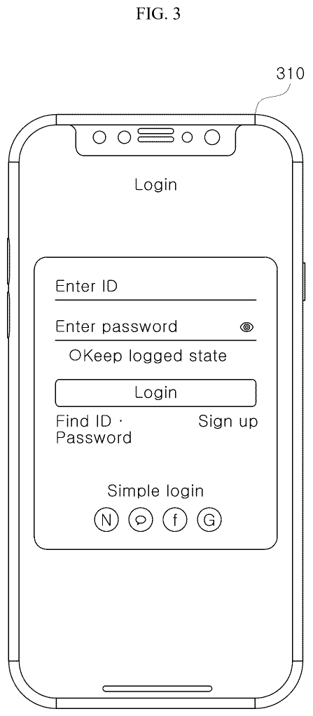

FIG. 3 is a diagram illustrating a start screen of an AI-based joint function analyzing and monitoring device, according to an embodiment of the present disclosure.

As shown in FIG. 3, when a specific application is executed, a start screen 310 includes an ID input button, a password input button, a login-state maintaining button, a login button, an ID·password finding button, a sign-up button, and four simple login buttons (Naver, Kakao, Facebook, and Google).

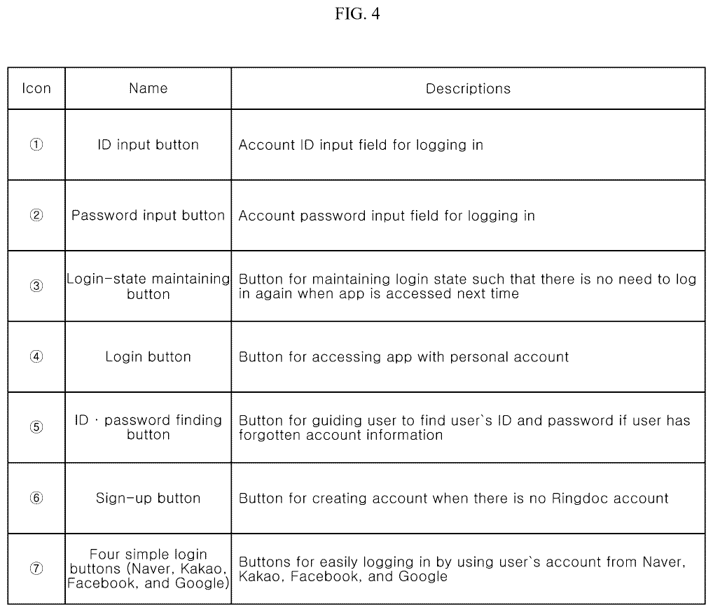

FIG. 4 is a drawing for describing a function of a start screen icon, according to an embodiment of the present disclosure.

Referring to FIG. 4, a function of a start screen icon is described.
1) The ID input button is an account ID input field for logging in.
2) The password input button is an account password input field for logging in.
3) The login-state maintaining button is a button for maintaining a login state such that there is no need to log in again when an app is accessed next time.
4) The login button is a button for accessing the app with a personal account.
5) The ID·password finding button is a button for guiding the user to find the user's ID and password if the user has forgotten account information.
6) The sign-up button is a button for creating an account when there is no first application account.

Here, the first application refers to an application that provides a service for AI-based joint function analysis.
7) The four simple login buttons (Naver, Kakao, Facebook, and Google) are buttons for easily logging in by using the user's account from Naver, Kakao, Facebook, and Google.

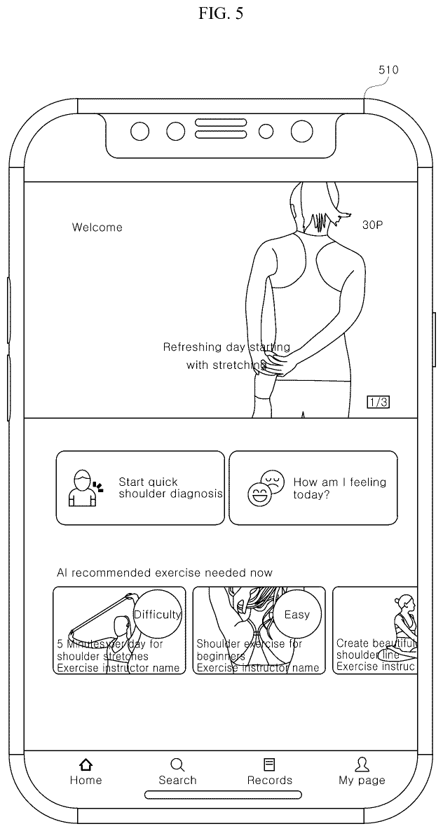

FIG. 5 is a drawing illustrating a menu screen, according to an embodiment of the present disclosure.

As shown in FIG. 5, the processor 130 displays a menu screen 510.

The menu screen (home screen) includes a screen for showing all services at a glance, including joint measurement, a mood state check, and an AI-recommended exercise. A joint state may be measured by pressing a ‘Start Quick Diagnosis’ button on the home screen.

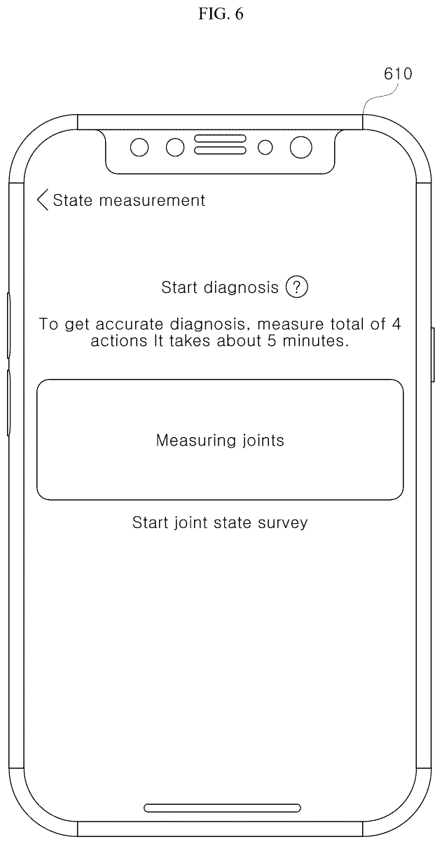

FIG. 6 is a diagram illustrating a joint-state evaluating screen, according to an embodiment of the present disclosure.

As illustrated in FIG. 6, the processor 130 displays a joint state evaluating screen 610.

The processor 130 starts by pressing a joint measuring button on a screen for checking an exact joint state.

The processor 130 measures a joint range of motion by pressing a measuring-joint button on the joint state measuring screen 610.

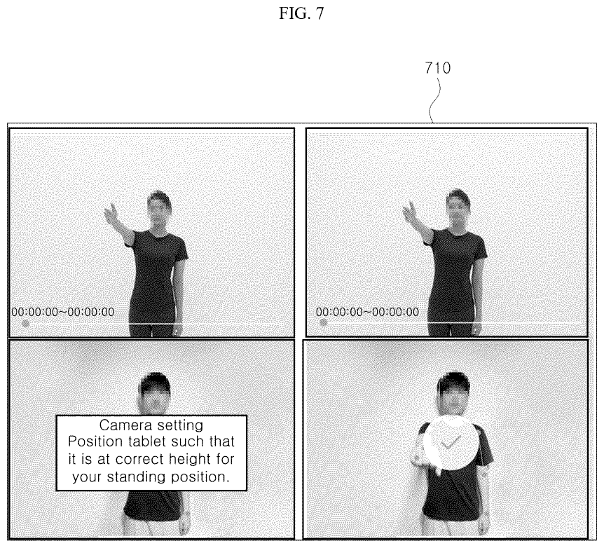

FIG. 7 is a diagram illustrating an example of a joint measuring screen (camera setting), according to an embodiment of the present disclosure.

As shown in FIG. 7, the processor 130 displays a joint measuring screen 710. The joint measuring screen 710 is a screen for measuring several joint angles in a correct pose.

Camera settings will be described. When the camera settings are completed, the several joint states are measured.

The processor 130 adjusts a camera such that the camera is in the correct measurable position for joint measurement.

The processor 130 sets the camera such that a screen illustrating the user standing is displayed while a mobile device is standing up.

FIG. 8 is a drawing illustrating an embodiment of a joint measuring screen, according to an embodiment of the present disclosure.

As shown in FIG. 8, the processor 130 displays a joint measuring screen 810.

The joint measuring screen 810 is a screen for providing feedback on whether a user is following the exercises recommended by a doctor.

When the joint image is a two-dimensional image, the processor 130 extracts the pose of a three-dimensional image based on the joint image and analyzes the joint angle of the three-dimensional image.

According to an embodiment of the present disclosure, the processor 130 may extract a 3D pose and analyze a joint angle by using a 2D image, thereby improving user convenience.

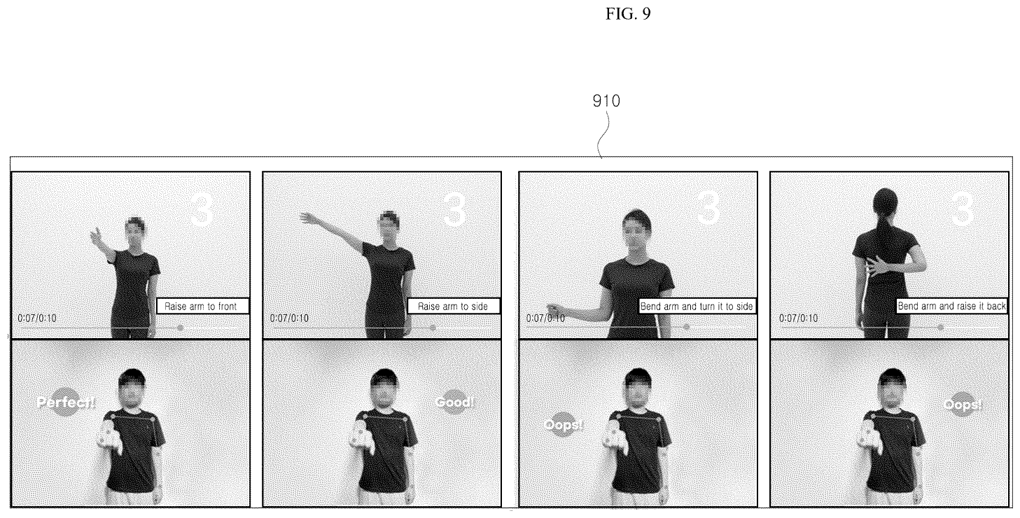

FIG. 9 is a drawing illustrating an embodiment of a joint measuring screen, according to an embodiment of the present disclosure.

As shown in FIG. 9, the processor 130 displays a joint measuring screen 910.

Referring to FIG. 9, when an exercise prescription is received, the processor 130 displays “perfect” when a patient follows it well, and displays “oops” when the patient does not follow it well.

FIG. 10 is a drawing illustrating an example of a survey screen, according to an embodiment of the present disclosure.

As shown in FIG. 10, the processor 130 displays a survey screen 1010.

The processor 130 identifies the degree of pain through the survey, and determines a pose in which the pain occurs, by asking questions about actions matching a direction in which the joint corresponding to the joint image moves.

The survey screen 1010 is a screen for conducting a survey on the function for a usual joint state after joint measurement.

The processor 130 completes the joint measurement by performing the final survey on functions.

The processor 130 determines the degree of pain and the pose in which the pain occurs.

The processor 130 determines the degree of pain through a questionnaire (question 1).

The processor 130 identifies the pose in which pain occurs, by asking questions about actions matching a direction in which a joint moves (questions 2 to 5).

Here, for the questionnaire, American Shoulder and Elbow Surgents Score that is internationally accepted is used.

Questions 2 to 7 are associated with the possible range of motion in daily life.

Questions 8 and 9 are associated with muscle strength.

Question 10 is a question for asking whether everyday tasks are possible in daily life.

Question 11 is a question for asking whether regular exercise and hobbies are possible in daily life.

The processor 130 collects the answers to the questions and then displays a graphic drawing a part, which is vulnerable, together with the total score as shown in the pentagram below.

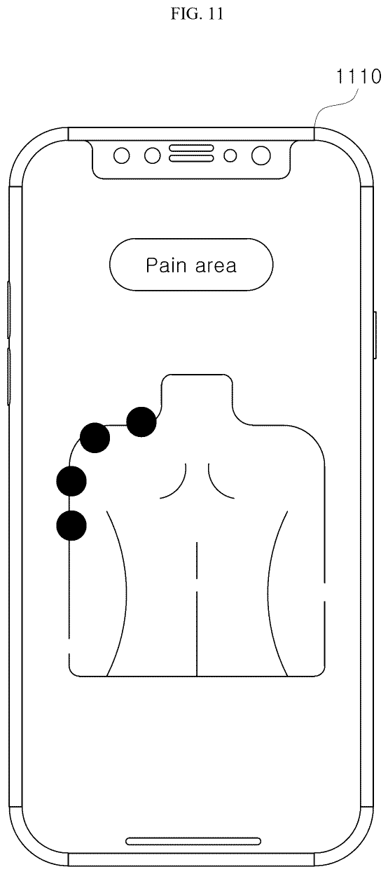

FIG. 11 is a drawing illustrating an embodiment of a pain area screen, according to an embodiment of the present disclosure.

As illustrated in FIG. 11, the processor 130 displays a pain area screen 1110.

The degree of pain and a pose in which the pain occurs may be identified through the questionnaire above, and the detailed location where the pain occurs may be identified in this way.

On the basis of the results of the descriptions above, the pose in which pain occurs may be displayed and the detailed location where the pain occurs may be displayed to a user.

For example, when the response to the question “Can you sleep on your sore shoulder?” is “uncomfortable,” the pain area may be clicked (or touched) by providing a body image such as the left of a body.

Besides, when there is a response of discomfort to the question “Is it difficult to wipe your back or, is it difficult to put on a bra in the case of women?”, a body image with arms bent toward the back may be provided, and the pain area may be clicked (or touched).

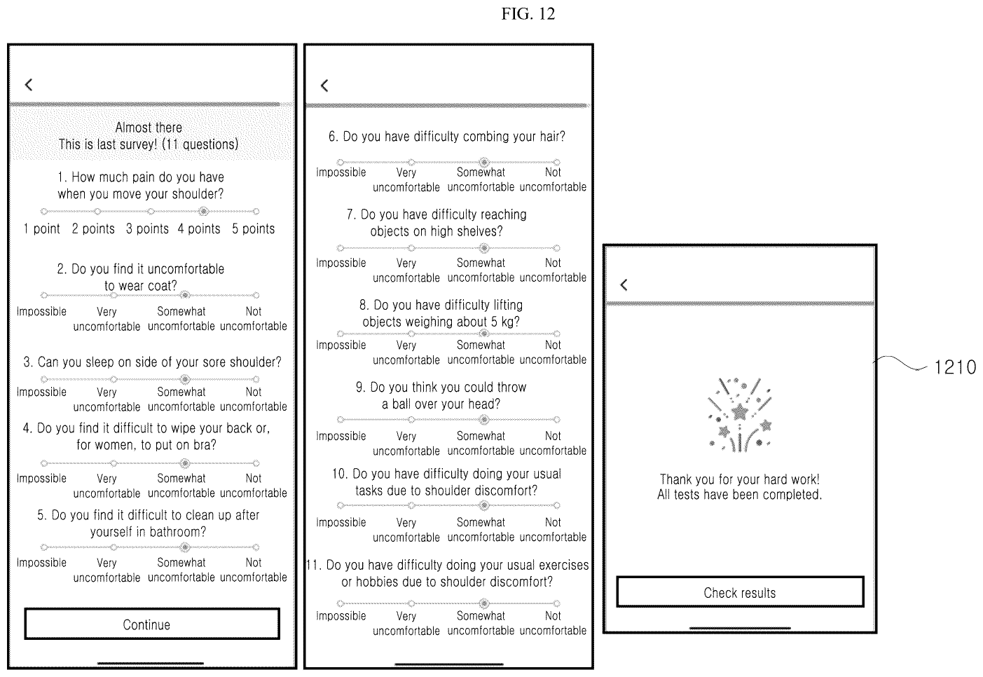

FIG. 12 is a drawing illustrating an example of a survey screen, according to an embodiment of the present disclosure.

As illustrated in FIG. 12, the processor 130 displays a survey screen 1210.

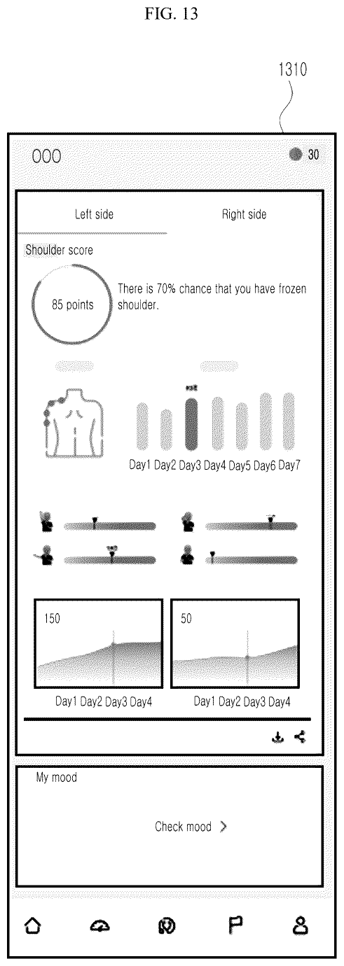

FIG. 13 is a diagram illustrating an embodiment of a state screen of a user, according to an embodiment of the present disclosure.

As illustrated in FIG. 13, the processor 130 displays a state screen 1310 of a user.

The processor 130 displays the state of the user after completing joint range measurement and function evaluation.

The processor 130 displays an image including a percentage of how much the user's state has improved from the first day of use.

When completing the joint range measurement and function evaluation, the processor 130 reflects feedback on which areas are vulnerable relative to age and gender, together with the total score for the user's state.

FIG. 14 is a diagram illustrating an example of a search screen, according to an embodiment of the present disclosure.

As illustrated in FIG. 14, the processor 130 displays a screen 1410 for searching for at least one of a doctor and a hospital corresponding to the pain area, and performs a search when receiving an input of a user.

In the case of doctor and hospital searches, it is possible to search for doctors and hospitals by searching for a pain area or a hospital in a search tab at the bottom of an application.

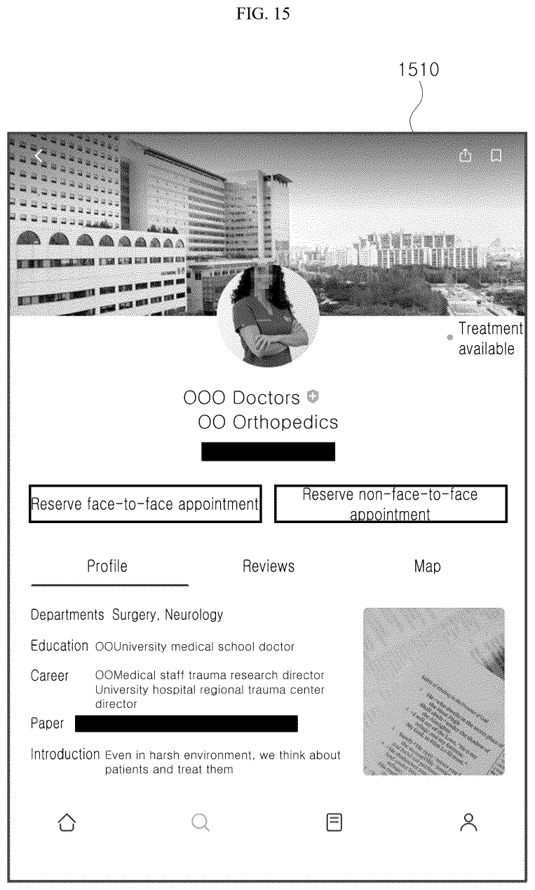

FIG. 15 is a diagram illustrating an embodiment of a doctor information screen, according to an embodiment of the present disclosure.

As illustrated in FIG. 15, the processor 130 displays a doctor information screen 1510.

The doctor information screen 1510 is a screen for identifying basic information about the doctor, reviews, hospital location, and the like and making an appointment for a face-to-face consultation.

FIG. 16 is a diagram illustrating an embodiment of a doctor treatment and customized exercise suggestion screen, according to an embodiment of the present disclosure.

As illustrated in FIG. 16, the processor 130 displays a screen 1610 for suggesting a customized exercise suggested by a doctor corresponding to a user's state.

During face-to-face treatment (or non-face-to-face treatment when telemedicine is permitted), a doctor may suggest personalized exercises and exercise frequency to a patient based on the recorded joint range of motion and the recorded functional evaluation through the suggestion screen 1610.

The suggestion screen 1610 is the doctor's web service screen for suggesting the most suitable exercise for each individual.

FIG. 17 is a diagram illustrating an example of a doctor suggestion motion screen, according to an embodiment of the present disclosure.

As illustrated in FIG. 17, when receiving a user selection input, the processor 130 displays the customized exercise image suggested by a doctor.

A patient may perform exercises at home according to the doctor's suggested exercises.

FIG. 18 is a diagram illustrating an embodiment of a doctor feedback record screen, according to an embodiment of the present disclosure.

As illustrated in FIG. 18, the processor 130 displays doctor information corresponding to a user's state, displays the measured joint range, and displays a record screen 1810 for identifying feedback from a doctor corresponding to the doctor information.

After joint measurement and treatment, the linked doctor information may be identified, and a record screen for monitoring the measured joint range and identifying feedback from the doctor may be displayed.

Doctor information, medical records, and doctor feedback, which are linked in the record tab at the bottom of an application, may be identified, and the measured joint range may be monitored.

FIG. 19 is a diagram illustrating an embodiment of My-Page, according to an embodiment of the present disclosure.

As illustrated in FIG. 19, the processor 130 displays a screen 1910 showing a user's treatment and appointment schedule.

The user may identify the user's activities such as treatment and reservation through the My-Page screen 1910.

FIG. 20 is a drawing illustrating an example of a chat execution screen, according to an embodiment of the present disclosure.

As illustrated in FIG. 20, the processor 130 displays a chat execution screen 2010 for chatting with a user.

Through chat between a first application coach and the user, the user's questions may be resolved and the common sense necessary for preventing and managing musculoskeletal disorders may be provided.

Here, the first application coach may be an artificial intelligence service server.

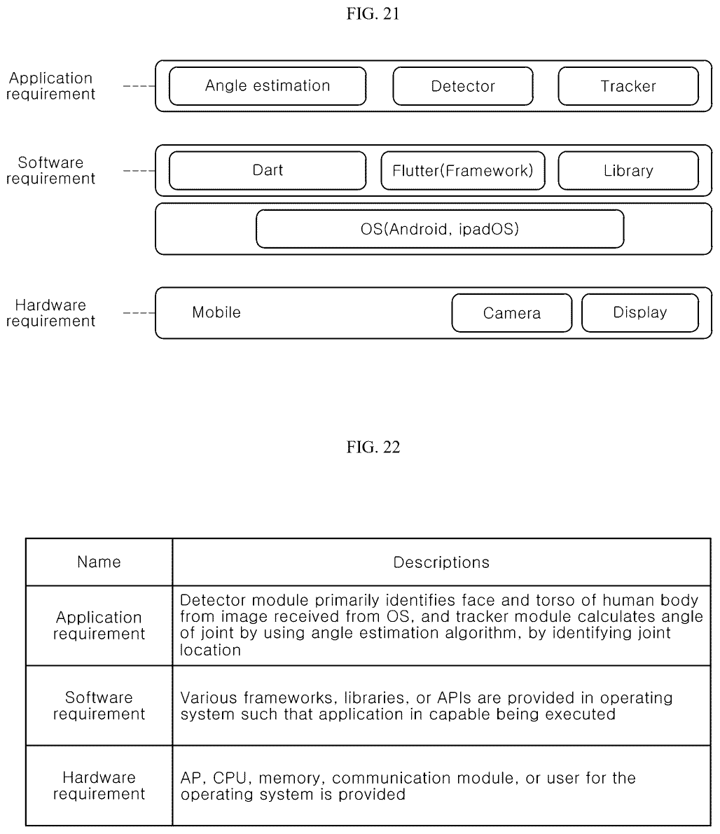

FIG. 21 is a drawing illustrating an embodiment of an operating system, according to an embodiment of the present disclosure.

As shown in FIG. 21, an operating system includes an application requirement, a software requirement, and a hardware requirement.

FIG. 22 is a drawing illustrating an example of a main description of an operating system, according to an embodiment of the present disclosure.

Descriptions will be given with reference to FIG. 22.
1) The application requirement indicates that a detector module primarily identifies the face and torso of a human body from an image received from OS, and a tracker module calculates the angle of a joint by using an angle estimation algorithm, by identifying a joint location.
2) The software requirement indicates that various frameworks, libraries, or APIs are provided in an operating system such that an application is capable of being executed.
3) The hardware requirement indicates that an AP, a CPU, a memory, a communication module, or a user interface for the operating system is provided.

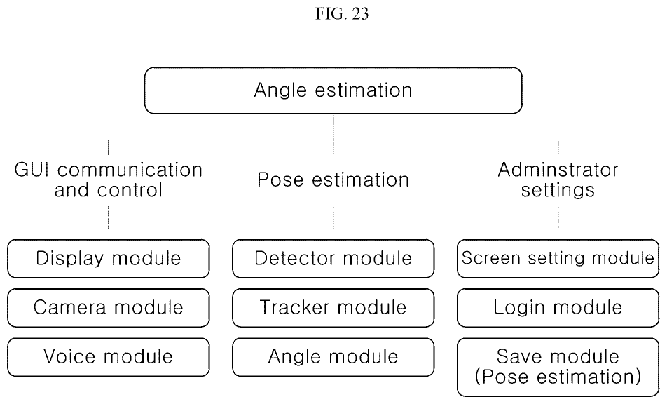

FIG. 23 is a diagram illustrating a software structure, according to an embodiment of the present disclosure.

The software architecture includes GUI communication and control, pose estimation, and administrator settings.

The GUI communication and control includes a display module, a camera module, and a voice module.

The pose estimation includes a detector module, a tracker module, and an angle module.

The administrator settings include a screen setting module, a login module, and a save module.

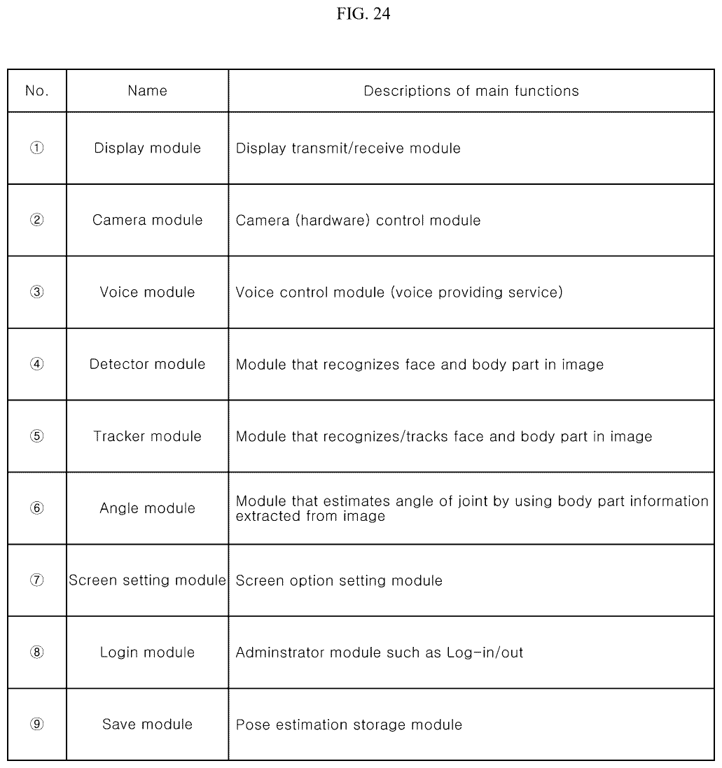

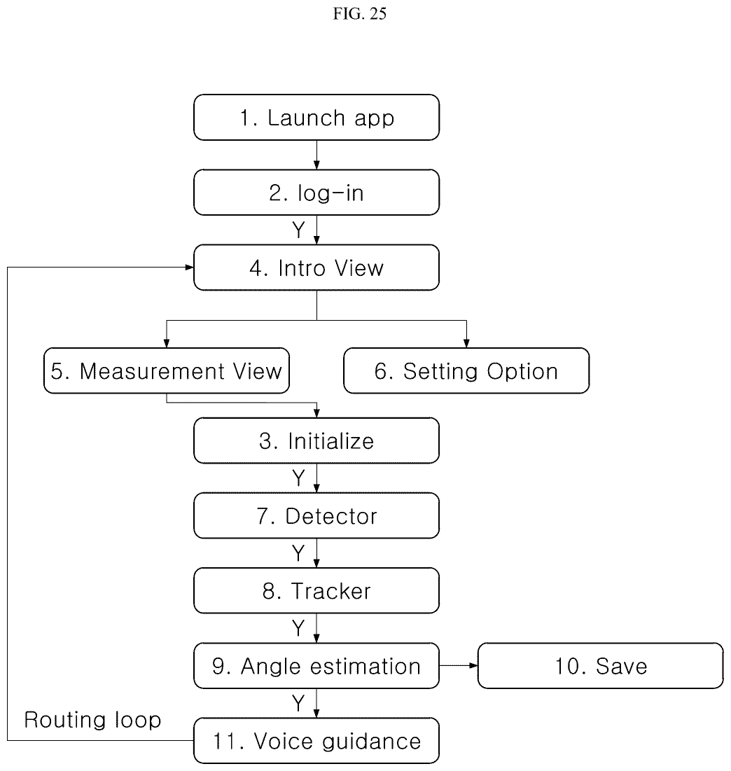

FIG. 24 is a diagram illustrating a main function of a software structure, according to an embodiment of the present disclosure.
1) The display module includes a display transmit/receive module.
2) The camera module includes a camera (hardware) control module.
3) The voice module includes a voice control module and a voice providing service.
4) The detector module is a module that recognizes a face and a body part in an image.
5) The tracker module is a module that recognizes/tracks a face and a body part in an image.
6) The angle module is a module that estimates the angle of a joint by using body part information extracted from an image.
7) The screen setting module is a screen option setting module.
8) The login module is an administrator module such as Log-in/out.
9) The save module is a pose estimation storage module.

FIG. 25 is a diagram illustrating a software algorithm, according to an embodiment of the present disclosure.

As shown in FIG. 25, an operating principle of Pose estimation using deep learning is described.

The present product is an application installed on the operating systems of Android or iPadOS of a mobile device. The range (angle) of motion is measured by extracting the location of each joint from the image of a camera operating in the OS through pose estimation using deep learning in the application.

In other words, extracting the joint angle of a three-dimensional pose based on the measured two-dimensional pose is the result of using the final operating principle, which estimates the joint angle for various poses based on only the frontal image of a person. Here, two modules are required to estimate a user's joint location, which are called a detector and a tracker. The detector first recognizes the head and torso of a person, which have relatively low degrees of freedom, to estimate an area where the joint of the person is likely to exist.

Afterwards, the tracker predicts a key point location of the joint to be determined in the area where the joint is likely to exist by the detector. After the key point location is predicted, the angle estimation module infers an angle between joints based on the location of each key point (joint) and a distance between the user and a camera.

A detector algorithm will be described.

The area where a joint is likely to exist is estimated by recognizing the head and torso of a person with low degrees of freedom in a frontal image of a person.

A tracker algorithm will be described.

The detector predicts the key point location of the joint to be determined in the area where the joint is likely to exist.

An angle estimation algorithm will be described.

The distance between the camera and the user and the location of each joint identified based on the tracker may be estimated, and the angle of the joint may be estimated based thereon.

A correction algorithm will be described.

There may be errors in results from deep learning. Accordingly, a user's body image (or skeleton) is corrected, and a value between the angle predicted based on the corrected result and the angle calculated through the deep learning is set as the final predicted value.

A methodology for reducing AI pose estimation errors will be described.

When errors that increase during a process of converting from 2D to 3D are corrected, errors of key points of joints inferred through the deep learning may increase during the process of converting from 2D to 3D. To solve the issues, the following steps are performed.

First, a user body shape analysis procedure is performed.

Before measuring a joint, it identifies the user's basic body shape such that the user matches the silhouette displayed on a screen.

This initial step is used as a basic reference for subsequent estimations.

Second, it stores a body shape-based 3D skeleton simulation and a lookup table.

A 3D skeleton is limited to a joint range in which a human is capable of moving based on a patient's body shape.

The simulation results of the skeleton for an instruction pose appearing in the prescription are stored in the lookup table.

Third, it integrates body shape data and a camera using the lookup table.

At least ‘n’ or more poses that are closest to the joint key point inferred from the deep learning are extracted based on the pre-simulated lookup table, and then similarity is calculated.

Afterwards, the camera and the body shape data are integrated by using an interpolation method via weighted average.

An error capable of occurring in single camera settings is accurately corrected through this process.

Fourth, it infers improved 3D joint key points.

The skeletal 3D joint key points for the user's pose may be more accurately inferred through this improved methodology.

This improved accuracy enables a reliable 3D representation of the user's movements.

Fifth, it measures a joint angle and makes a final prediction.

It may accurately measure the angle between joints based on these refined joint key points.

This measurement ensures relatively high accuracy in a process of predicting the user's pose.

This modified methodology provides a systematic and effective approach to minimizing errors in AI pose estimation. The purpose of use will be described.

It corresponds to software that measures the joint range of motion using AI, evaluates a joint function, recommends the most suitable exercise for each individual based on the data, and provides a service to receive monitoring from a doctor.

A storage method will be described.

The user's joint angle measured through an application is transmitted from the user's mobile device to a server connected to the application by using a HTTP protocol so as to be stored.

FIG. 26 is a diagram illustrating a comparison group and a functional recovery speed analysis, according to an embodiment of the present disclosure.

As illustrated in FIG. 26, the processor 130 receives detailed information including the user's gender and age, calculates a comparison target based on the detailed information, and displays a functional recovery speed comparing and analyzing result 2610 of comparing the calculated comparison target with the functional recovery speed.

A technology for comparing with a comparison group will be described.

The technology receives necessary information.

The technology receives information about the user's gender, age, artificial joint replacement, or the like.

In the case of artificial joint replacements, the technology may compare the degree of recovery according to the type of replacement. However, the technology may also identify how quickly or slowly recovery occurs compared to people of the same age and gender after various surgeries and diseases, such as frozen shoulder and rotator cuff repair. The technology of the present disclosure may also be applied to knees.

The technology calculates a comparison target (or comparison group) based on the user's detailed information such as gender, age, and artificial joint, or directly receives the comparison target (or comparison group).

The functional recovery speed comparison will be described.

The technology perform comparison with the functional recovery speed of a comparison target (or comparison group) and identifies what needs to be improved by a user by analyzing differences in rehabilitation programs or exercise volume, exercise angles, and the like.

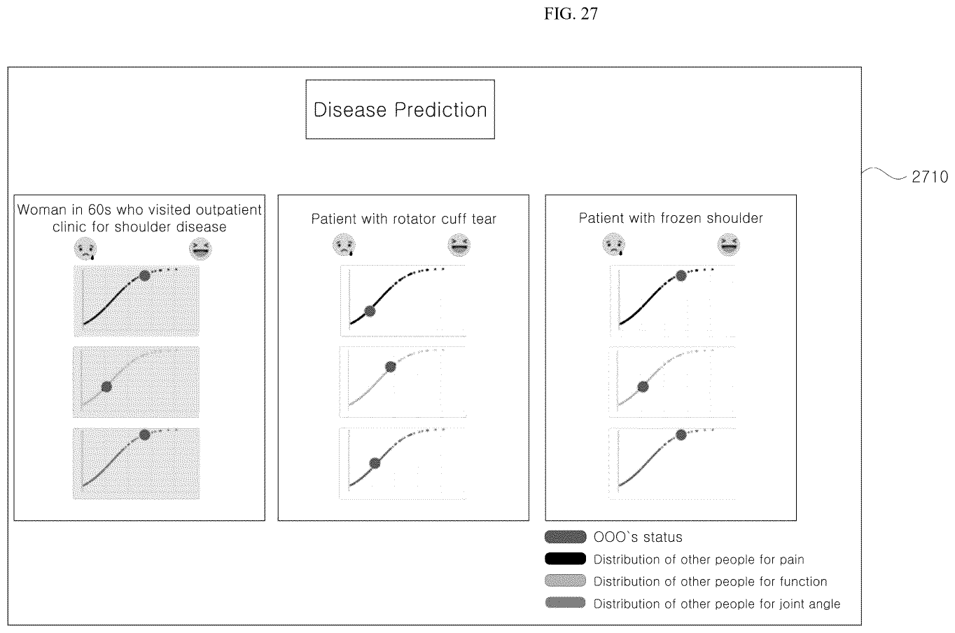

FIG. 27 is a diagram illustrating an example of predicting a disease through comparison with other patients, according to an embodiment of the present disclosure.

As illustrated in FIG. 27, the processor 130 receives detailed information of a woman in 60s who visited an outpatient clinic for shoulder disease, a patient with a rotator cuff tear, and a patient with frozen shoulder, predicts the disease based on the detailed information, and displays the prediction result (2710).

According to an embodiment of the present disclosure, a user's disease may be predicted through comparison with other disease patients in terms of pain, function, joint range, joint distortion, muscle strength, etc.

The present disclosure expresses the symptom similarity between a user and a person with each disease on a graph.

For example, the possibility of predicting rotator cuff tear through logistic regression was analyzed to be approximately 84% by analyzing the function and joint range of each of approximately 200 patients.

Additionally, the present disclosure inputs a neural network by using Explainable AI, increases a prediction rate, and describes the reason that an AI made such the prediction.

The AI of the conventional technology gives the answer, but fails to explain the process of why it came to that answer. On the other hand, the present disclosure overcomes this limitation.

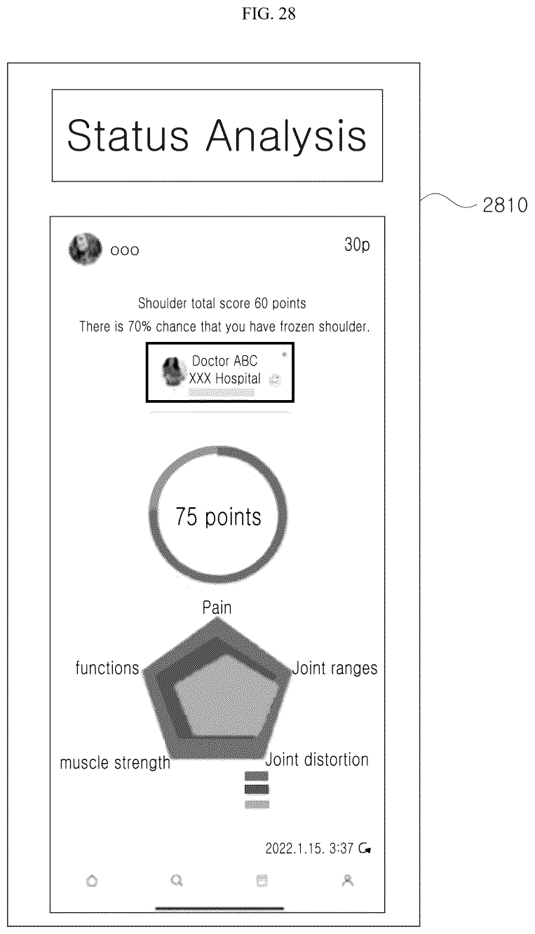

FIG. 28 is a diagram illustrating state analysis of a user, according to an embodiment of the present disclosure.

As illustrated in FIG. 28, the processor 130 displays a user state analysis screen 2810.

The processor 130 analyzes states by scoring pain, functions, joint ranges, joint distortion, and muscle strength.

Here, the muscle strength is measured via separate hardware operating in conjunction with a first application.

In detail, the muscle strength may be replaced with questions associated with muscle strength from among questions in a survey. Afterwards, when hardware is developed, feedback may be provided with more accurate numbers.

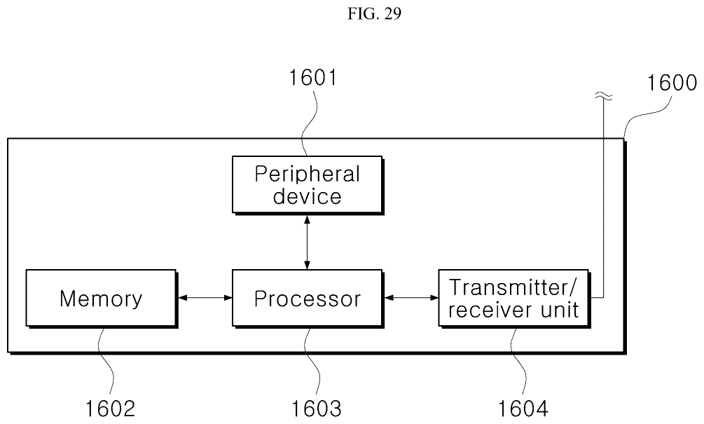

FIG. 29 is a diagram illustrating a configuration of an AI-based joint function analyzing and monitoring device, according to an embodiment of the present disclosure.

Referring to FIG. 29, the present disclosure includes a device 1600. The device 1600 may include a memory 1602, a processor 1603, a transmitter/receiver unit 1604, and a peripheral device 1601. Moreover, for example, the device 1600 may further include other configurations and is not limited to the above-described embodiments.

In more detail, the device 1600 of FIG. 29 may have hardware/software architecture such as an NDN device, an NDN server, a content router, or the like. In this case, for example, the memory 1602 may be a non-removable memory or a removable memory. Besides, for example, the peripheral device 1601 may include a display, GPS, or other peripherals, and is not limited to the above-described embodiments.

Furthermore, for example, the device 1600 described above may include a communication circuit, such as the transmitter/receiver unit 1604, and may communicate with an external device based thereon.

Furthermore, for example, the processor 1603 may be at least one or more of a general purpose processor, a digital signal processor (DSP), a DSP core, a controller, a microcontroller, Application Specific Integrated Circuits (ASICs), Field Programmable Gate Array (FPGA) circuits, any other type of integrated circuit (IC), and one or more microprocessors associated with a state machine. That is, it may be a hardware/software configuration that performs a control role for controlling the device 1600 described above.

In this case, the processor 1603 may execute computer-executable instructions stored in the memory 1602 to perform various essential functions of the present disclosure. For example, the processor 1603 may control at least one of signal coding, data processing, power control, input/output processing, and communication operations. In addition, the processor 1603 may control a physical layer, an MAC layer, and application layers. Also, for example, the processor 1603 may perform authentication and security procedures at an access layer and/or an application layer, and is not limited to the above-described embodiment.

For example, the processor 1603 may communicate with other devices through the transmitter/receiver unit 1604. For example, the processor 1603 may allow a node to communicate with other nodes over a network by executing the computer-executable instructions. That is, the communication performed by the present disclosure may be controlled. For example, the other nodes may be an NDN server, a content router, and other devices. For example, the transmitter/receiver unit 1604 may transmit an RF signal via an antenna and may transmit a signal based on various communication networks.

Moreover, for example, MIMO technology, beamforming, or the like may be applied as antenna technology, and is not limited to the above-described embodiment. Furthermore, the signal transmitted and received through the transmitter/receiver unit 1604 may be modulated and demodulated and may controlled by the processor 1603, and may not be limited to the above-described embodiment.

According to the above-mentioned problem solving means of the present disclosure, a user's state may be inferred based on the user's joint image, the results of a survey and the pain area, and the user state result may be displayed, and thus the user may intuitively know the user's condition, thereby improving the user convenience.

According to the above-mentioned problem solving means of the present disclosure, an app capable of diagnosing a user's state may automatically operate in conjunction with a computer system and may efficiently deliver the user's state to medical staff, thereby improving user convenience.

According to the above-mentioned problem solving means of the present disclosure, scattered personal health data may be collected in a standardized way and utilized for personal health management, and data through the personal health data may be used to extract the association between chronic diseases and musculoskeletal diseases in the future to present the most suitable life log for the individual, thereby improving user convenience.

According to the above-mentioned problem solving means of the present disclosure, additional value based on big data may be created by operating in conjunction with data between hospitals, thereby improving user convenience.

Various embodiments of the present disclosure are not intended to list all possible combinations, but are intended to describe representative aspects of the present disclosure, and the matters described in the various embodiments may be applied independently or in two or more combinations.

What is claimed is:

1. An artificial intelligence (AI)-based joint function analyzing and monitoring device comprising:
an input module configured to receive a user input;
a display module configured to display a graphic image;
a memory configured to store at least one process for performing an operation and to store the user input and data;
a camera module configured to capture an image in front; and
a processor configured to perform a AI-based joint function analysis operation according to the process,
wherein the processor is configured to:
allow the display module to display a start screen for receiving user login from a user;
display a menu screen for receiving an entire service including joint measurement, a mood state check, and AI-recommended exercise of the user;
display a joint measurement screen when receiving a joint measurement input from the user;
capture a joint image of the user taking a plurality of poses on the joint measurement screen by using the camera module;
display a survey screen for a joint state of the user, and receive an input for a survey from the user;
display an image for displaying a pain area of the user corresponding to an answer to the survey, and receive an input for the pain area from the user;
infer a state of the user based on the joint image, a result of the survey, and a result of the pain area; and
display the state of the user.

2. The AI-based joint function analyzing and monitoring device of claim 1, wherein the processor is configured to:
when the joint image is a two-dimensional image, extract a pose of a three-dimensional image based on the joint image; and
analyze a joint angle of the three-dimensional image.

3. The AI-based joint function analyzing and monitoring device of claim 1, wherein the processor is configured to:
identify a degree of pain through the survey; and
identify a pose in which pain occurs, by asking a question about an action matching a direction in which the joint corresponding to the joint image moves.

4. The AI-based joint function analyzing and monitoring device of claim 1, wherein the processor is configured to:
display a screen for searching for at least one of a doctor and a hospital corresponding to the pain area; and
perform a search when receiving an input of the user.

5. The AI-based joint function analyzing and monitoring device of claim 1, wherein the processor is configured to:
display a screen for suggesting a customized exercise suggested by a doctor corresponding to the state of the user.

6. The AI-based joint function analyzing and monitoring device of claim 1, wherein the processor is configured to:
when receiving a user selection input, display a customized exercise image suggested by a doctor.

7. The AI-based joint function analyzing and monitoring device of claim 1, wherein the processor is configured to:
display doctor information corresponding to the state of the user;
display a joint range corresponding to the joint image; and
display a record screen for identifying feedback from a doctor corresponding to the doctor information.

8. The AI-based joint function analyzing and monitoring device of claim 1,
wherein the processor is configured to:
display a screen for showing a treatment and an appointment schedule of the user.

9. The AI-based joint function analyzing and monitoring device of claim 1, wherein the processor is configured to:
display a chat execution screen for chatting with the user.

10. An AI-based joint function analyzing and monitoring control method, the method comprising:
displaying a start screen for receiving user login from a user;
displaying a menu screen for receiving an entire service including joint measurement, a mood state check, and AI-recommended exercise of the user;
displaying a joint measurement screen when receiving a joint measurement input from the user;
capturing a joint image of the user taking a plurality of poses on the joint measurement screen by using a camera;
displaying a survey screen for a joint state of the user, and receiving an input for a survey from the user;
displaying an image for displaying a pain area of the user corresponding to an answer to the survey, and receiving an input for the pain area from the user;
inferring a state of the user based on the joint image, a result of the survey, and a result of the pain area; and
displaying the state of the user.
